(19) United States
(12) Patent Application Publication
(10) Pub. No.: US 2021/0309973 A1
(43) Pub. Date: Oct. 7, 2021

(54) Bacteriophage-Based Antibodies and Binders

(71) Applicant: ATHANOR BIOSCIENCES, INC., HALETHORPE, MD (US)

(72) Inventors: HOSSEIN A. GHANBARI, POTOMAC, MD (US); MICHAEL S. LEBOWITZ, PIKESVILLE, MD (US)

(21) Appl. No.: 17/222,463

(22) Filed: Apr. 5, 2021

Related U.S. Application Data
(60) Provisional application No. 63/005,342, filed on Apr. 5, 2020.
(60) Provisional application No. 63/073,905, filed on Sep. 2, 2020.

(51) Int. Cl. C12N 7/00; A61K 35/76; C07K 16/10; C12N 9/48; C07K 14/71

(52) U.S. Cl. CPC C12N 7/00; A61K 35/76; C07K 16/10; C12N 9/485; C12Y 304/17023; C07K 2319/00; C12N 2795/00021; C12N 2795/00052; C12N 2795/00032; C07K 2317/622; C07K 14/71

(57) ABSTRACT

Engineered bacteriophage and methods of forming the bacteriophage are described. Multivalent bacteriophage are described that can include multiple different exogenous polypeptides that include specific binding agents for proteinaceous targets at a surface of the capsid head. Therapeutic compositions, e.g., antiviral compositions, and methods of forming are described. A therapeutic composition can include an engineered bacteriophage that includes a polypeptide binds a pathogen or binds a cellular receptor of a pathogen at a surface of the bacteriophage. The engineered bacteriophage are free of nucleic acids encoding the exogenous polypeptide(s).

### CROSS REFERENCE TO RELATED APPLICATION

This application claims filing benefit of U.S. Provisional Patent Application Ser. No. 63/005,342, entitled, “Super-Antibodies and Super-Binders,” to Ghanbari et al., having a filing date of Apr. 5, 2020, which is incorporated herein by reference; and claims filing benefit of U.S. Provisional Patent Application Ser. No. 63/073,905, entitled, “Super-Antibodies and Super-Binders,” to Ghanbari et al., having a filing date Sep. 2, 2020, which is incorporated herein by reference.

### BACKGROUND

Disease processes include infectious diseases and auto-maladies. Infectious diseases are caused by micro-organisms, such as bacteria, fungi, and parasites, as well as by viruses. These agents can infect higher order eukaryotes, including humans, where they replicate within the host tissues. While many of these infectious agents are innocuous and even beneficial to the normal function of the host organism, many cause disease with symptoms that can range from mild to severe and, in some instances, may result in the death of the host organism. Auto-maladies are diseases caused by self-agents, rather than infectious agents (auto vs. xeno). Cancer is a prominent auto-malady. Other examples are autoimmune diseases, blood-borne diseases, etc.

Of particular interest among infectious diseases are viral diseases. Viruses are essentially non-living infectious agents consisting of a nucleic acid (DNA or RNA) encapsulated in a viral coat, often made from protein, glycoprotein and/or lipid. Upon transfer to the host organism, the virus binds to and obtains access to specific cells where it can utilize the host cellular machinery to replicate and produce large numbers of new viral particles. These new viral particles are subsequently released from the infected cell, often by lysis of the cell, and go on to infect other host cells. The viral DNA or RNA generally encodes for relatively few viral-specific proteins including structural proteins, proteins necessary for cellular attachment and entry as well as any viral specific enzymes required for replication (e.g., reverse transcriptase, integrase and/or a protease). Some viruses can integrate into the host genome and lie dormant for long periods of time, only to re-emerge much later in the host cell's lifetime. Viruses cause disease by disrupting normal cellular functions and often inducing strong inflammatory responses within the host organism. They may also carry toxins and/or transfer genetic sequences into the host cell's chromosomes.

The mammalian immune system is specially adapted to respond to viral infections. The immune system consists of three major arms: the innate immune system, the humoral immune response, and the cellular immune response. The innate immune response is initiated through a process called pattern recognition and includes the activation of a series of general protective measures affording initial protection from infection (e.g., inflammation) and promoting the activation of more specific responses (e.g., activation of professional antigen presenting cells). The humoral response evolves against a specific pathogen and involves the production by B-cells of immunoglobulins or antibodies with high specificity towards the pathogen. Antibodies are blood proteins produced by the immune system in response to foreign antigens that have exquisite specificity for their targeted antigens. Antibodies will bind to specific antigens on the pathogen, coating it, and resulting in its clearance from the host tissues. In the case of infected cells, the antibodies can attach to the infected host cell and activate the complement system to kill the infected cell. The cellular response consists of the activation of highly specific T-cells, including both cytotoxic and helper T-cells. Cytotoxic T-cells directly recognize infected host cells and initiate apoptotic mechanisms in them. Helper T-cells support the activation and maturation of both pathogen-specific B-cells and cytotoxic T-cells.

Because of their relatively high binding affinities/avidities, their high specificity for target and the fact that they are natural mammalian proteins, antibodies have been employed as drugs in instances where their binding properties would be advantageous. Antibodies by nature are at least bivalent as they contain two equivalent binding sites for antigens and thus potentially increase overall avidity when the target antigen is presented in a multimeric format as may occur on a cell surface. Avidity of antibody-based drugs have been further increased by using IgM in which five antibody molecules are linked to yield overall 10 antigen combining sites.

Bacteriophage (or more simply phage) are viruses that infect bacterial cells. These viruses consist of a protein coat which encapsulates a DNA or RNA genome. When phage infect a bacterial cell, they can coopt the host bacterial system to produce large numbers of phage copies and ultimately lyse the bacterial cell, releasing the new phage to the surrounding environment.

Bacteriophage have been used to display peptide or protein fragments for various uses. For example, phage display systems have been used to map the epitopes of antibodies or to identify single chain fragments of antibodies (scFv) that bind to specific antigens. These phage display systems gain their selection power in part from the ability to display many copies of a protein on the surface of the phage. By way of example, when using bacteriophage lambda (A), the displayed protein is often engineered as an extension of the phage gpD coat protein.

This ability of phage to present on their surfaces large numbers of a protein or protein fragment qualifies them as bio-nanoparticles (BNPs). The use of phage as BNPs has been especially advantageous as compared to other synthetic nanoparticles or bio-nanoparticles as phage are simple to genetically engineer and are easy to purify. Furthermore, phage can be produced at extremely high-yield in readily available bio-fermenters. As such, the development and manufacture processes can be rapid and highly cost-effective. In vivo, phage are known to have long half-lives and their size allows for easy tissue penetration. In general, phage have demonstrated only low levels of immunogenicity in mammals, including humans, likely due to early exposure and partial acquired immune tolerance due to the abundance of phage in the natural environment.

While the above describes improvement in the art, room for added improvement exists. For instance, phage for use in applications, such as antiviral applications, could be of great benefit in the art.

### SUMMARY

According to one embodiment, disclosed is an engineered bacteriophage. The engineered bacteriophage includes multiple specific binding agents that can be the same or different from one another at a surface of the phage. More specifically, an engineered bacteriophage can include fusion coat proteins that in turn include exogenous polypeptides directly or indirectly fused to one or more coat proteins of the bacteriophage, which can be of the same or different types of coat proteins. The exogenous polypeptides can include one or more sequences which are specific binding agents for one or more proteinaceous targets. For instance, an engineered bacteriophage can include a first fusion coat protein that includes a first exogenous polypeptide fused to a first coat protein and a second fusion coat protein that includes a second exogenous polypeptide fused to a second coat protein. The first and second exogenous polypeptides can include first and second specific binding agents that can be the same or different from one another and the first and second coat proteins of the fusion coat proteins can likewise be the same or different from one another. The engineered bacteriophage can also be free of nucleic acid sequences encoding the exogenous polypeptide(s).

The target of a specific binding agent of an engineered bacteriophage can be a viral target, e.g., a cellular receptor for a viral pathogen or a coat protein and/or other protein of a viral pathogen. Thus, the engineered phage can block binding between a viral pathogen and a host cell. In other embodiments, a target of a specific binding agent can be of a different type of pathogen or any other binding target of a disease process. For instance, an engineered bacteriophage can be an anti-bacterial agent, an anti-fungal agent, an anti-parasitic, can target an auto-immune pathogen, e.g., a cancer cell, or can function as a protein inhibitor, among other applications.

Also disclosed are therapeutic compositions, e.g., an antiviral composition, that can include an engineered bacteriophage in which the bacteriophage includes multiple fusion coat proteins, each of which can include an exogenous polypeptide that includes a specific binding agent fused to a coat protein of the bacteriophage. The binding agents of the fusion coat proteins can be the same or different from one another and the coat proteins of the fusion coat proteins can be the same or different from one another. A therapeutic composition can also generally include a delivery system, e.g., a delivery vehicle suitable for delivery via inhalation, injection, implantation, etc.

Also disclosed is a method for forming an engineered bacteriophage. For instance, a method can include transfecting a bacterial cell with an expression plasmid and infecting the bacterial cell with a phage. The expression plasmid can include a nucleic acid sequence that encodes a fusion coat protein. The fusion coat protein can include an exogenous polypeptide directly or indirectly fused to a coat protein of the phage. The exogenous polypeptide can include a specific binding agent for a proteinaceous target. The expression plasmid can also include regulatory sequences such that following the transfection, the fusion coat protein is transiently expressed by the bacterial cell. Upon the infection and the transfection, an engineered phage can be produced by the bacterial cell that includes the fusion coat protein and that is free of nucleic acid sequences encoding the exogenous polypeptide. The methods can also include incorporation of one or more additional nucleic acids encoding one or more different fusion coat proteins on a single expression plasmid or transfecting the bacterial cell with one or more additional expression plasmids that include nucleic acid sequence(s) encoding one or more additional fusion coat proteins. Thus, a method can form a multivalent phage with high affinity and binding avidity for one or more targets.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying FIGURES in which:

FIG. 1 schematically illustrates one embodiment of a multivalent bacteriophage as described herein.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

Disclosed herein are engineered bacteriophage and methods of forming the bacteriophage as well as therapeutic compositions including the bacteriophage. The engineered bacteriophage include one or more specific binding agents for one or more proteinaceous targets involved in a disease process. For instance, and as further described herein, specific binding agents of an engineered phage can target a protein of a pathogen or a protein binding target of a pathogen. Thus, an engineered phage can prevent activity of a pathogen in a host. Targeted pathogens can be of any type, e.g., invasive targets such as viral, bacterial, or fungal pathogens, or targets that arise within a host, e.g., targets related to autoimmune or other disease processes.

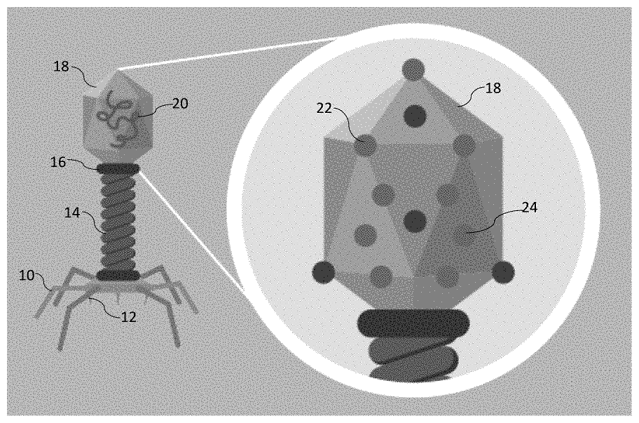

In one embodiment, an engineered bacteriophage can be a multivalent bacteriophage and can include multiple different exogenous polypeptides that each contain one or more specific binding agents at the surface of the capsid head. FIG. 1 schematically illustrates one embodiment of a multivalent bacteriophage. As illustrated, a multivalent bacteriophage can include typical bacteriophage components including tail fiber 10, spikes 12, and a sheath 14. A collar 16 typically separates the sheath 14 from the capsid head 18, which encases the bacteriophage DNA 20. The capsid head 18 is formed from a plurality of coat proteins, e.g., gpD, gpE and gpC coat protein in the case of bacteriophage A. Multivalent bacteriophage as disclosed herein can include two or more different fusion coat proteins 22, 24 in the capsid head 18 that each include an exogenous polypeptide at a terminal end of a coat protein. Different fusion coat proteins 22, 24 can differ from one another by difference(s) in the exogenous polypeptide portion of the fusion coat proteins, difference(s) in the coat protein portion of the fusion coat proteins, or both.

For instance, different engineered bacteriophage can include two or more different fusion coat proteins in which the different fusion coat proteins include the same exogenous polypeptide at the end of different coat proteins of the phage. For instance, a first fusion coat protein can include an exogenous polypeptide that includes a first specific binding agent directly or indirectly bonded to a terminal end of a first type of coat protein of the phage, and a second fusion coat protein can include the same exogenous polypeptide directly or indirectly bonded to a terminal end of a second, different coat protein of the phage. Methods of forming such engineered bacteriophage are also described.

An engineered bacteriophage can also include combinations of the above, e.g., multiple different fusion coat proteins that include combinations of fusion coat proteins with one or more different exogenous polypeptides including one or more different specific binding agents directly or indirectly bonded to terminal ends of one or more different types of coat protein of the phage. Moreover, in some embodiments, a single fusion coat protein of an engineered phage can carry multiple specific binding agents, which can be the same or different from one another and that can be directly or indirectly bonded to one another, with the multiple specific binding agents directly or indirectly bonded to a terminal end of the coat protein.

Also disclosed are therapeutic compositions incorporating an engineered bacteriophage. More specifically, a therapeutic composition can include an engineered bacteriophage that exhibits one or more exogenous polypeptides that each include one or more specific binding agents for a proteinaceous target at a surface of the bacteriophage. The bacteriophage can also be free of nucleic acid encoding the exogenous polypeptide(s).

In one embodiment, a therapeutic composition can include a multivalent bacteriophage that can include multiple different exogenous polypeptides that include specific binding agents that target various different proteinaceous targets involved in a disease process, e.g., a viral infection. The binding agents can target the same or different proteins involved in the disease. For instance, the binding agents can incorporate a natural protein binding partner of a pathogen or a mimic thereof. For example, to engage a receptor for a pathogen, a natural ligand can be employed as a specific binding agent of an engineered phage or in the reverse to engage a ligand a mimic of the receptor can be employed as a specific binding agent of an engineered phage. Binding of a ligand by a soluble form of a receptor can be used to sequester the ligand and prevent its engagement of its conjugate receptor. Binding of a receptor by a mimic of a ligand can be used to block the engagement of receptor by the natural ligand.

In one embodiment, different binding agents of a bacteriophage can specifically bind a single protein target, e.g., different peptide sequences of a single protein involved in a disease. For example, a first exogenous polypeptide can include a binding agent specific for a first targeted location of a protein of a pathogen (e.g., a first binding site of a viral coat protein) and a second exogenous polypeptide can include a second binding agent specific for a second location of the same protein (e.g., a second binding site of the same viral coat protein). In one embodiment, different binding agents of a bacteriophage can specifically bind different proteins of the same pathogen. For instance, a first binding agent can specifically bind a first coat protein of a viral pathogen and a second binding agent can bind a different coat protein of the same viral pathogen. Of course, any combination of binding agents are encompassed herein as well.

As previously mentioned, protein targets of an engineered bacteriophage can also include proteins that are not those of the pathogen itself, but rather host proteins targeted by a pathogen. For instance, a specific binding agent of an exogenous polypeptide can specifically bind a cell surface receptor that is the binding target of a pathogen, thereby blocking the pathogen from binding the cell.

In one embodiment a therapeutic composition can include a multivalent bacteriophage that includes multiple different exogenous polypeptides designed to specifically bind multiple different proteins of disease caused by different pathogens, e.g., different variants of a virus type. In one embodiment, a therapeutic composition can include a multivalent bacteriophage that includes one or more different exogenous polypeptides that can specifically bind targets from different phases of a disease process. For instance, an exogenous polypeptide can include a specific binding agent for a protein of a pathogen, e.g., an antibody or binding fragment thereof specific for a coat protein of a viral pathogen. Such a binding agent can be effective at blocking the pathogen prior to the presentation of any disease symptoms. The same or a different exogenous polypeptide of the engineered phage can include another specific binding agent for a protein involved in the disease process, e.g., an antihistamine, an anti-inflammatory, or the like, that can be effective against the initial host response to the pathogen. Combinations of different types and binding targets of exogenous polypeptides can also be incorporated in a multivalent bacteriophage included in a therapeutic composition as described.

Disclosed therapeutic compositions can be particularly effective as anti-viral compositions as they can incorporate high affinity and highly specific antibody sequences against a particular target antigen.

Viruses are important and potentially deadly human pathogens. These infectious agents can jump species and rapidly spread amongst local populations and given the advances made in transportation and global human mobility can be spread throughout the world creating health crises even pandemics. Over the past several decades the world has witnessed such crises develop in the wake of SARS, MERS, and most recently, COVID-19 (SARS-Cov-2). Even common viruses such as influenza remain major health risks for human populations and result in significant numbers of deaths per year. One therapeutic approach is to prevent viral entry into cells. Many viruses that infect mammalian cells are enveloped viruses; meaning that the viral particle is enclosed in a phospholipid bilayer. This bilayer generally incorporates several different types of proteins, including the viral coat protein that is generally involved attachment of the virus to the cell and subsequent entry into the cell. Many copies of this coat protein are maintained on the surface of the virus. As such, one way to prevent viral entry into the cell is to block the viral coat protein by engaging it with either a neutralizing antibody or a soluble form of the cellular receptor. While in certain instances, such approaches have shown efficacy, the effectiveness may be limited due either low affinities of the engager or the sheer number of binding events that must occur to completely block viral entry.

Bacteriophage have a number of unique characteristics that make them a superior platform for a wide variety of applications, including in an anti-viral application. For instance, the ability to display large copy numbers of exogenous polypeptides on the surface of a phage allows for improved effectiveness due to the large numbers of binding agents that can be displayed on the phage surface. Simply put, when two relatively large entities interact, for example two cells or a cell and a virus, the existence of multiple engagement points can greatly enhance the overall strength of the interaction (avidity). By way of example, when using bacteriophage lambda, the displayed exogenous polypeptide can be engineered as an extension of the phage gpD coat protein. 400 copies of the gpD protein are used by the phage to construct its coat and as such up to 400 copies of the exogenous protein can be displayed on the phage surface. Moreover, the exogenous polypeptides can be quite large. For instance, in the case of bacteriophage lambda, relatively large proteins (300 amino acids or more) or multiple binding agents that are the same or different from one another ligated in a single polypeptide can be displayed without disrupting the ability of the phage coat to form.

The ability to display large copy numbers of polypeptides on the surface of the phage allows for the construction of binding entities that can be highly multivalent and that can bind with much greater binding avidity as compared monovalent binders or to standard IgG antibodies, which have only two combining sites, or even to IgM which has maximally 10 binding sites. Furthermore, the ability to display multiple different binding entities simultaneously on the surface of the phage allows for the ability to interact with the target in multiple ways. These “Super-Antibodies” or “Super-Binders” can display hundreds of copies of one or more specific binding partners, and as such, can bind to cells, viruses, or other targets with extremely high avidity.

Beneficially, disclosed formation methods can provide for phage that do not incorporate any foreign DNA or RNA, and as such, there is no concern of transfer of DNA or RNA to a subject, which can be particularly beneficial in anti-viral applications. The natural phage themselves are generally non-infective to mammalian cells and due to their abundance in nature, the mammalian immune system is likely pre-exposed to the natural phage and potentially even tolerized against anti-phage responses. Moreover, as the phage are bio-nanoparticles, they can optionally be irradiated prior to use to prevent any potential infectivity to a subject, for instance infectivity of symbiotic host prokaryotic organisms.

Disclosed engineered phage can include at least one exogenous polypeptide at a surface of the phage coat. As utilizes herein, the term “exogenous” refers to a material that originates external to and is not naturally found as a component of either the phage or the bacterial cell that is used to produce the engineered phage. As utilized herein, the term “polypeptide” generally refers to a polymeric molecule including two or more amino acid residues, which can include natural and synthetic amino acids as well as combinations thereof and includes proteins as well as fragments. As utilized herein, the term “fragment” generally refers to a continuous part of a full-length protein, with or without mutations, which is separate from and not in the context of a full length protein. A fragment may be a structural/topographical or functional subunit of a full length protein. In some embodiments, a fragment can have an amino acid sequence of about 15 or more amino acids, or about 20 or more amino acids of the parent full-length surface protein.

In one embodiment, a binding agent of an exogenous polypeptide can be developed from a protein or a protein target of a pathogen. For instance, an exogenous polypeptide can include one or more neutralizing single-chain antibodies (scFv) and/or mimics of cellular receptors of viral coat proteins. For example, a binding agent can be identical to (e.g., correspond to) a known antibody of a pathogenic protein or a binding fragment thereof or can be a functional mutant or homologue of an antibody or an antibody fragment. As utilized herein, the term “homologue” generally refers to a nucleotide or polypeptide sequence that differs from a reference sequence by modification(s) that do not affect the overall functioning of the sequence. For example, when considering polypeptide sequences, homologues include polypeptides having substitution of one amino acid at a given position in the sequence for another amino acid of the same class (e.g., amino acids that share characteristics of hydrophobicity, charge, pK or other conformational or chemical properties, e.g., valine for leucine, arginine for lysine, etc.). Homologues can include one or more substitutions, deletions, or insertions, located at positions of the sequence that do not alter the conformation or folding of a polypeptide to the extent that the biological activity of the polypeptide is destroyed. Examples of possible homologues include polypeptide sequences and nucleic acids encoding polypeptide sequences that include substitution of one non-polar (hydrophobic) residue such as isoleucine, valine, leucine or methionine for another; the substitution of one polar (hydrophilic) residue for another such as between arginine and lysine, between glutamine and asparagine, or between threonine and serine; the substitution of one basic residue such as lysine, arginine or histidine for another; the substitution of one acidic residue, such as aspartic acid or glutamic acid for the another; or the use of a chemically derivatized residue in place of a non-derivatized residue, as long as the homolog displays substantially similar biological activity to the reference sequence.

Formation methods of disclosed bacteriophage include transfecting a bacterial cell with an expression plasmid that includes a nucleic acid sequence encoding a fusion phage coat protein. An expression plasmid can be produced by recombinant DNA technology as known. A fusion coat protein encoded by the expression plasmid can include a native or wild type phage coat protein with an added N- or C-terminal extension (e.g., a full single chain antibody or receptor mimic or fragment thereof) that can be directly or indirectly fused to a terminus of the coat protein (e.g., indirectly fused by inclusion of a spacer between the two or by inclusion of additional full chain binding agent proteins or fragments thereof on a single coat protein). An expression plasmid can be transfected into the bacterial cell prior to or in conjunction with infection of the bacterial cell with a phage that naturally includes the coat protein of the fusion coat protein encoded by the expression plasmid.

An expression plasmid can include a DNA sequence encoding a phage coat protein ligated to a DNA sequence encoding the selected exogenous polypeptide(s) such that the exogenous polypeptide sequence(s) is in frame with the coat protein sequence. DNA encoding a short linker sequence may be placed between the sequences if desired, for instance to achieve successful expression.

The coat protein encoded in the expression plasmid and expressed with an exogenous polypeptide as a fusion coat protein can vary depending upon the phage type. The phage may be any bacteriophage known to those skilled in the art, including, but not limited to, λ, M13, T4, T7, φX174.

By way of example, when forming an engineered A phage, an expression plasmid can include DNA encoding one or more fusion coat proteins based on one or more of the gpD, gpE or gpC coat proteins in conjunction with the encoding of one or more exogenous polypeptides in any combination. For example DNA of one or more plasmids can encode a first exogenous polypeptide in conjunction with a gpD coat protein, as well as encoding that same exogenous polypeptide in conjunction with a gpE coat protein, DNA of one or more plasmids can encode a first exogenous polypeptide in conjunction with a gpD coat protein as well as a second, different exogenous polypeptide in conjunction with a gpD coat protein, can encode a first exogenous polypeptide in conjunction with a gpD coat protein as well as a second, different exogenous polypeptide in conjunction with a different, e.g., gpE, coat protein, or any combination thereof.

If an engineered M13 phage is to be formed, one or more of the pVIII, pill, pVI, pVII or pIX proteins can generally be encoded in an expression plasmid in conjunction with one or more exogenous polypeptides. Similarly, the gp23 and/or gp24 proteins can generally be encoded when forming an engineered T4 bacteriophage and the gp10A and/or gp10B proteins can be encoded in an expression plasmid when forming an engineered T7 phage. For phage φX174 the gpF and/or gpG proteins can generally be encoded in conjunction with one or more exogenous polypeptides.

A hybrid DNA sequence encoding a fusion coat protein can be placed into a bacterial expression plasmid under the control of a suitable bacterial expression promoter. A promoter can be an inducible promoter, a copy of a native phage promoter or any promoter deemed appropriate by one skilled in the art. The expression plasmid can be one that provides for transient expression of the fused coat protein in the bacterial cell. Transient expression systems have been used as tools of recombinant technology for many years and as such is not described in detail herein. By way of example and without limitation, suitable transient expression systems can include the pETDuet™ family of vectors from Novagen/EMD Millipore.

In forming a multivalent bacteriophage, in one embodiment, a single expression plasmid can include multiple different hybrid DNA sequences, each of which encode a different fusion coat protein. For instance, in one embodiment, an expression plasmid can include one or more variant copies of a hybrid DNA sequence, each of which encoding a different exogenous polypeptide extension of the same fusion coat protein. The different exogenous polypeptide sequences can be different variants of a natural polypeptide (e.g., corresponding to the natural polypeptide and one or more homologues or mutants of the natural polypeptide) or different polypeptides all together. In another embodiment, multiple different plasmids may be used in forming a multivalent bacteriophage, with different plasmids including different hybrid DNA sequences that encode for a different fusion coat protein (e.g., different by the exogenous polypeptide extension, the phage coat protein, or both).

In another embodiment, an engineered bacteriophage may display only a single exogenous polypeptide sequence, but may include multiple different fusion coat proteins, with the exogenous polypeptide sequence as a component of different fusion coat proteins that incorporate different types of coat proteins of the native phage.

When using different expression plasmids to carry different fusion coat protein DNA, the regulatory components of the expression plasmids can be the same or differ from one another. For instance, in one embodiment, different expression plasmids can be essentially the same as one another other than the fusion coat protein DNA sequences. In one embodiment, different selection markers can be incorporated on the different expression plasmids, which can be used to ensure that selected production bacteria have incorporated all plasmid types. In one embodiment, different plasmids or different expression components of a single plasmid can incorporate different promotors driving expression of the fusion coat proteins, for instance, different strength promoters, thus allowing for the fusion coat proteins with different exogenous polypeptide extensions to be produced at varying levels which can also allow for incorporation of the different fusion coat proteins into an engineered phage at different ratios.

Exogenous polypeptide sequences chosen for inclusion in a fusion coat protein may be derived from any source and can include complete proteins, protein fragments, mutants, or homologues thereof. In one embodiment, polypeptide sequences chosen for display may be derived from any protein known to bind to a pathogen specific (e.g., a non-host pathogen such as a virus) protein. In anti-viral applications, the polypeptides sequences can bind to sequences found in the viral coat proteins but may also bind to other major proteins present in the virus. These displayed proteins may correspond to antibodies or any derivative thereof including but not limited to single-chain antibodies (scFv) and/or nanobodies (VH or VHH sequences). Displayed proteins may also correspond to host receptor proteins or any derivative thereof.

In one embodiment, binding agent sequences can be derived from known neutralizing antibodies produced in individuals previously known to have had immune responses to the virus, where this information is available. Sequences may bind to a single strain, multiple strains of a certain family and/or different families of pathogens, e.g., viral pathogens.

DNA sequences incorporated in an expression plasmid can encode an exogenous polypeptide of any length. For instance, a DNA sequence of an expression plasmid can encode an exogenous polypeptide that is about 12 amino acids or greater in length, for instance about 15 amino acids or greater, about 50 amino acids or greater, about 100 amino acids or greater, or about 150 amino acids or greater in length in some embodiments. In one embodiment, a DNA sequence of an expression plasmid can encode an exogenous polypeptide that is about 500 amino acids or less in length, for instance about 450 amino acids or less, about 400 amino acids or less, about 350 amino acids or less, or about 300 amino acids or less in length in some embodiments. The exogenous polypeptide of a fusion coat protein can be of any length provided it does not interfere with the incorporation of the fusion coat protein in the bacteriophage during formation thereof by the bacterial cell.

In one embodiment a multivalent bacteriophage can be engineered that can include multiple different fragments (or homologues thereof) of a single protein, for instance, when the natural protein of interest is large and incorporation of the entire protein sequence in a single fusion coat protein could interfere with bacteriophage formation.

It should be understood that exogenous polypeptides are not limited to those for use in an anti-viral application, and other exogenous polypeptide types are encompassed herein. By way of example, an exogenous polypeptide can be developed to bind a pathogen or a protein involved in a disease related to a pathogen including, and without limitation to, bacterial pathogens, fungal pathogens, parasites, and/or viral pathogens, as well as self-rising disease processes that do not necessarily involve an invasive pathogen. Invasive viral pathogens encompassed herein can include, without limitation, coronavirus, influenza, HIV, HCV, HBV, HPV, dengue, Chikungunya, and West Nile.

By way of example, in one particular embodiment, an engineered bacteriophage can be a component of an anti-viral therapeutic against a coronavirus. Four major membrane surface proteins are known to be expressed on the surface of coronavirus virus particles, S, E, M, and N. In one embodiment, binding agents for all four of these proteins and/or mimics of cellular receptors of the viral coat proteins can be used to develop exogenous polypeptides on one or more different phage or on a single phage. In one embodiment one or more other types of coronavirus proteins can be used to develop a binding agent of an exogenous polypeptide, for instance in conjunction with one or more binding agent of a surface protein.

In the case of the coronaviruses, the primary viral coat protein is referred to as the spike or S-protein and in the specific case of SARS-CoV-2 the cellular receptor for the spike protein is angiotensin converting enzyme 2 (ACE2). In one embodiment, an engineered bacteriophage can express one or more scFv targeted at SARS-CoV-2 coat proteins on the surface of bacteriophage lambda. The phage, expressing many copies of each scFv on their surfaces, can serve as extremely high avidity neutralizing antibodies against SARS-CoV-2, preventing viral entry and likely due to the size of the complex leading to clearance of the coronavirus from the system by phagocytic cells. By expressing more than one scFv on the surface of the phage, the phage can effectively supply the equivalent of a polyclonal antibody response to the coronavirus.

In one exemplary embodiment, ACE2 can be expressed on the surface of the bacteriophage lambda. This can produce a multivalent receptor mimic with potentially greater affinity/avidity for SARS-CoV-2 over the natural cell surface ACE2 receptor. This exemplary engineered bacteriophage can bind to SARS-CoV-2 preventing viral entry into cells and again, likely due to the size of the complex, leading to clearance of the coronavirus from the system by phagocytic cells. Similar approaches could be taken to target other pathogens and proteins involved in a disease process.

For example, a multivalent engineered bacteriophage can include binding agents at the surface directed to one or more proteins involved in an infection by one, multiple, or all known coronaviruses including, without limitation, SARS (e.g., SARS-CoV-1, SARS-CoV-2), MERS, HKU (e.g., HKU1), NL63, OC43, and/or 229E. Proteins as may be utilized in development of an anti-viral therapeutic as described herein can include, without limitation, human coronavirus 229E surface glycoprotein (accession no. NP_073551.1), human coronavirus NL63 S protein (accession no. AFV53148.1), human coronavirus HKU1 spike glycoprotein (accession no. YP_173238.1), human coronavirus OC43 S protein (accession no. QDH43726.1), Middle East respiratory syndrome-related (MERS) coronavirus S protein (QFQ59587.1), SARS coronavirus Urbani S protein (accession no. AAP13441.1), and severe acute respiratory syndrome coronavirus surface glycoprotein (accession no. 2YP_009724390.1).

Upon development of one or more expression plasmids that include DNA encoding the one or more fusion coat proteins, the plasmid(s) can be transfected into a host bacterial cell. The host bacterial cell can be any suitable type that is also infectable by the phage that is to be the basis for the engineered phage product. For instance, when forming an engineered bacteriophage A, the host bacterial cell can be an E. coli and an E. coli can thus be transfected with the expression plasmid(s) according to standard transfection practice. Suitable bacterial hosts for phage infection are known to those in the art.

In conjunction with or subsequent to the transfection of the host with the one or more expression plasmid(s), the host can be infected with the phage of choice. Depending upon the transfection/expression system utilized, additional components as necessary can be supplied to the bacterial host. For instance, if an inducible promoter is incorporated in the expression plasmid(s), the inducing agent can also be supplied to the bacterial host during phage infection.

Upon transfection and infection, the bacterial host can produce the engineered bacteriophage that incorporate the fusion coat proteins. Beneficially, because the fusion coat proteins are produced from plasmid(s) transiently expressed in the bacteria during phage production, the DNA encoding the exogenous polypeptide is not incorporated into the phage.

The amount of fusion coat proteins incorporated into an engineered bacteriophage can be controlled in one embodiment, for instance through selection of the promoter strength of an expression plasmid. Such an approach can be used to control relative amount of different fusion coat protein in a bacteriophage as well as relative amount of the natural, e.g., wild type, coat protein vs. the fusion coat protein. In such an embodiment, the natural phage coat protein upon which the fusion coat protein is based can be maintained to a controlled extent on the engineered phage. Thus, the engineered phage can include a portion of the coat protein lacking any fused exogenous polypeptide in addition to the fused coat protein.

In one embodiment, the bacterial cell can be infected with a knock-out phage in which the wild-type coat protein expression has been silenced or deleted. In this case, all of the coat protein of the type incorporated in the expression plasmid (e.g., all gpD coat protein of a bacteriophage A) can be present in the expressed engineered phage as fusion coat protein.

Following transfection and infection, a host bacteria can be grown until lysis of the bacteria. Once bacterial cell lysis has occurred, the phage can be purified and characterized using standard techniques. It should be noted that loss of infectivity by the modified phage is not a problem for the use of the engineered phage and can be advantageous in some embodiments, for instance in an anti-viral application.

Engineered phage as described can serve as bio-nanoparticles that are easily manufactured in bacterial cultures and that can be grown at large scale in standard bio-fermenters.

Following lysis, engineered bacteriophage may be purified by any number of methods known to those skilled in the art for bacteriophage purification. These methods include, but are not limited to polyethylene glycol (PEG) precipitation, tangential flow filtration, affinity chromatography, etc. Engineered bacteriophage may be further concentrated, devoided of bacterial endotoxins, and characterized by standard methods known to those skilled in the art.

In one embodiment, an engineered bacteriophage can include one or more components at the surface that can prevent immune reactivity of the phage in a host. In one embodiment, control of immunogenicity can be achieved by including in the phage fusion proteins that include the IL-10 protein such that the IL-10 protein is expressed on the surface of the engineered phage. IL-10 is known to suppress antigen presentation by professional antigen presenting cells (APCs) including dendritic cells and macrophages. By supplying IL-10 on the phage, rather than systemically, only APCs that are likely to endocytose and present phage derived proteins could be suppressed rather than suppression of all APC function.

A therapeutic composition formulation can be carried out to include the bacteriophage according to known formation protocols as are known to those skilled in the art. For instance, purified bacteriophage can be transferred into a buffered saline solution with commonly used preservatives and filter sterilized. Because of the high stability of bacteriophage, a therapeutic composition incorporating an engineered bacteriophage can be stable at ambient and room temperatures for long periods, e.g., one week to several months.

A therapeutic composition can be prepared in one embodiment as an injectable, either as a liquid solution or suspension. A solid form suitable for solution in, or suspension in, liquid prior to injection may also be prepared. The preparation may also be emulsified, or the ingredients can be mixed with excipients that are pharmaceutically acceptable and compatible with the bacteriophage. Suitable excipients are, for example, saline or buffered saline (pH 7 to 8), or other physiologic, isotonic solutions that may also contain dextrose, glycerol or the like and combinations thereof. In addition, a therapeutic composition can contain minor amounts of auxiliary substances such as wetting or emulsifying agents, pH buffering agents that can enhance the effectiveness of the vaccine.

A therapeutic composition can be prepared in one embodiment as an inhalable composition. For instance, an inhalable therapeutic composition can include the engineered bacteriophage as individual particles or as a component of a larger particle or droplet in which the particle/droplet size facilitates penetration throughout the lungs. In one embodiment, a therapeutic composition designed to be inhaled from a dry powder inhaler can include dry particles comprising engineered bacteriophage as described. In one embodiment, an inhalable composition can include particles or droplets comprising engineered bacteriophage suspended in a propellant, e.g., in the form of an aerosol. In one embodiment, an inhalable composition can be a suspension of droplets or particles comprising engineered bacteriophage held in a liquid carrier that can be intended for administration by use of a liquid nebulizer system. In such an embodiment, a therapeutic composition can incorporate an aqueous liquid carrier, a nonaqueous liquid carrier, or can include a combination of an aqueous and nonaqueous carrier.

A pharmaceutical composition can include individual particles or droplets having a size that can permit penetration into the alveoli of the lungs, generally about 10 μm or less in size, about 7.5 μm or less in size, or about 5 μm or less in size in some embodiments. For instance, when considering aerodynamically light particles (e.g., having a bulk density of about 0.5 g/cm³ or less) for delivery as a dry powder formulation, a pharmaceutical composition can carry larger particles, for instance having a size of from about 5 μm to about 30 μm.

A therapeutic composition can be delivered by any of the standard routes including but not limited to intramuscular, intravenous, subcutaneous, intradermal, inhalation, etc. A therapeutic composition can be applied or instilled into body cavities, absorbed through the skin (e.g., via a transdermal patch), inhaled, ingested, topically applied to tissue, or administered parenterally via, for instance, intravenous, peritoneal, or intraarterial administration.

A delivery device can be utilized that allows controlled or sustained release, such as a sponge, biocompatible meshwork, mechanical reservoir, or mechanical implant. Implants and devices as can be useful for administration of a therapeutic composition have been described and are known in the art (see, e.g., U.S. Pat. Nos. 5,443,505 and 4,863,457, both of which are incorporated by reference herein). A therapeutic composition can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

An engineered phage can be used to control and/or treat existing disease and or prophylactically to prevent disease when an individual is concerned about being exposed to a pathogen.

The dosage of a therapeutic composition administered to a subject can depend on a number of factors, including the extent of any side-effects, the particular route of administration, and the like. The dose ideally comprises an “effective amount” of a therapeutic composition, i.e., a dose of the binding agent polypeptide carried on an engineered bacteriophage that can prevent activity of the pathogen of interest or otherwise interfere with the disease process.

### Example 1

In this example a bacteriophage is formed to display a single scFv, either the scFv CR3014 (CR3014 Heavy Chain (Accession # AAT51715), Light Chain (Accession # AAT51718)) or scFV CR3022 (CR3022 Heavy Chain (Accession #ABA54613), Light Chain (Accession #ABA54614)). In either case the heavy and light chains of the individual scFv are linked via the sequence GGGGSGGGGSGGGGS (SEQ ID NO: 1). Both of these scFv have been previously identified as neutralizing antibodies against the Spike protein of SARS-CoV-1 and have been shown to bind to the spike protein of SARS-CoV-2.

DNA encoding these scFv were engineered such that the DNA sequence was attached to the 3′ end of DNA encoding the lambda phage gpD protein with a short piece of DNA in between which codes for the amino acid sequence, GGSGPVGPGGSGAS (SEQ ID NO: 2).

The engineered protein was expressed in bacteria simultaneously with lambda infection of the same bacterium. These bacteria produced new lambda phage which incorporate the gpD-linker-scFv fusion protein together with natural phage gpD. The complete sequence for CR3014 is:

| SEQ ID NO: 3 | | |
|---|---|---|
| 1 | MTSKETFTHYQPQGNSDPAHTATAPGGLSAKAPAMTPLMLDTSSRKLVAW | 50 |
| 51 | DGTTDGAAVGILAVAADQTSTTLTFYKSGTFRYEDVLWPEAASDETKKRT | 100 |
| 101 | AFAGTAISIVGGSGPVGPGGSGASEVQLVESGGGLVQPGGSLRLSCAASG | 150 |
| 151 | FTFSDHYMDWVRQAPGKGLEWVGRTRNKANSYTTEYAASVKGRFTISRDD | 200 |
| 201 | SKNSLYLQMNSLKTEDTAVYYCARGISPFYFDYWGQGTLVTVSSGGGGSG | 250 |
| 251 | GGGSGGGGSELTQSPSSLSASVGDRVTITCRASQSISSYLNWYQQKPGKA | 300 |
| 301 | PKLLIYAASSLQSGVPSRFSGSGSGTDFTLTISSLQPEDFATYYCQQSYS | 350 |
| 351 | TPPTFGQGTKVEIK | 364 |

Where amino acids 1-110 encode for gpD, amino acids 111-124 (italic) encode for a linker (SEQ ID NO: 2), amino acids 125-244 (bold) encode for the heavy chain variable region of CR3014, amino acids 245-259 (italic) encode for a linker (SEQ ID NO: 1) and amino acids 260-364 (bold) encode for the light chain variable region of CR3014. The complete sequence for CR3022 is:

| SEQ ID NO: 4 | | |
|---|---|---|
| 1 | MTSKETFTHYQPQGNSDPAHTATAPGGLSAKAPAMTPLMLDTSSRKLVAW | 50 |
| 51 | DGTTDGAAVGILAVAADQTSTTLTFYKSGTFRYEDVLWPEAASDETKKRT | 100 |
| 101 | AFAGTAISIVGGSGPVGPGGSGASQMQLVQSGTEVKKPGESLKISCKGSG | 150 |
| 151 | YGFITYWIGWVRQMPGKGLEWMGIIYPGDSETRYSPSFQGQVTISADKSI | 200 |
| 201 | NTAYLQWSSLKASDTAIYYCAGGSGISTPMDVWGQGTTVTVSSGGGGSGG | 250 |
| 251 | GGSGGGGSDIQLTQSPDSLAVSLGERATINCKSSQSVLYSSINKNYLAWY | 300 |
| 301 | QQKPGQPPKLLIYWASTRESGVPDRFSGSGSGTDFTLTISSLQAEDVAVY | 350 |
| 351 | YCQQYYSTPYTFGQGTKVEIK | 371 |

Where amino acids 1-110 encode for gpD, amino acids 111-124 (italic) encode for a linker (SEQ ID NO: 2), amino acids 125-243 (bold) encode for the heavy chain variable region of CR3022, amino acids 244-258 (italic) encode for a linker (SEQ ID NO: 1) and amino acids 259-371 (bold) encode for the light chain variable region of CR3022.

The nucleic acid sequence encoding the gpD-CR3014 fusion protein was determined from the amino acid sequence and was optimized for codon usage in E. coli K12. The nucleic acid sequence for the gpD-CR3014 fusion protein was cloned into the expression vector pACYCDuet-1™ (Novagen) in the second multi-cloning site at the restriction site for NdeI. The resultant plasmid was sequenced across all junctions to ensure proper construction. Expression was under a T7 promoter. The final vector, designated pACYC-CR3014, was transfected into E. coli BL21 (DE3). Resultant phage produced in E. coli expressing the gpD-CR3014 represent super-antibodies and are designated ATHR-B5.

Alternatively, the nucleic acid sequence encoding the fusion protein gpD-CR3022 was determined from the amino acid sequence and was optimized for codon usage in E. coli K12. The nucleic acid sequence for the gpD-CR3022 fusion protein was cloned into the expression vector pACYCDuet-1™ (Novagen) in the first multi-cloning site at the restriction site for NcoI. The resultant plasmid was sequenced across all junctions to ensure proper construction. Expression was under a T7 promoter. The final vector, designated pACYC-CR3022, was transfected into E. coli BL21 (DE3). Resultant phage produced in E. coli expressing the gpD-CR3022 represent super-antibodies and are designated ATHR-B6.

Expression of ATHR-B5 or ATHR-B6 was achieved as follows: E. coli transfected with the pACYC-CR3014 or pACYC-CR3022 expression vector were selected on chloramphenicol containing medium, grown to an OD₆₀₀=0.4-0.8 at 37° C., induced with IPTG and simultaneously infected with wildtype bacteriophage λ (ATCC 23724 B2) at an MOI of 1-10. Cultures were allowed to grow for 4-5 hours.

Phage were isolated by centrifugation of cultures at 10000×g to remove cells and cell debris. The supernatant was clarified through a 0.45p filter and then a 0.22p filter and then concentrated ˜25-fold and buffer exchanged into PBS using tangential flow filtration (TFF) through a Minimate™ TFF Capsule 500 k Omega™ Membrane (PALL).

Removal of endotoxin was achieved with ethanol precipitation followed by precipitation with Triton™ X-100. Ethanol was added to the retentate to 30% and the material was allowed to precipitate overnight at room temperature. The precipitate was removed by centrifugation and filtration through a 0.22p filter. PBS was added to the remaining filtered supernatant to reduce the ethanol concentration to 25% and the bulk material was again processed through TFF using the Minimate™ TFF Capsule 500 k Omega™ Membrane. The material was diafiltered with 4 times the volume of PBS+25% Ethanol followed by 4 times the volume of PBS. The volume of the retentate was subsequently reduced by 2-fold to approximately 1/50 of the original culture volume. The retentate was cooled to 4° C. and Triton™ X-114 was added to 0.33-0.67% (v/v). The material was slowly warmed to ˜37° C. while centrifuged to remove the precipitate. The final material was concentrated and washed with PBS using a Macrosep® ADV 100 KD filtration device to a volume ˜ 1/100 of the original culture volume.

ATHR-B5 and ATHR-B6 were biochemically characterized. The particle count was established using the NanoSight NS300 Instrument (Malvern Panalytical) with nanoparticle tracking analysis (NTA) software to establish the size and count of bionanoparticles. Dot blots, Western blots and sandwich ELISAs with antibodies against human antibody sequences were used to establish expression of the scFv on the surface of the phage. The functionality of the displayed scFv was demonstrated by binding to recombinant Spike-RBD protein by ELISA.

### Example 2

In this example both CR3014 and CR3022 were displayed simultaneously on the surface of phage λ.

The expression vector was designed as above in example 1 except that both the gpD-CR3022 and the gpD-CR3014 sequences were placed into the same vector with the gpD-CR3022 cloned into the NcoI site and the gpD-CR3014 cloned into the NdeI site. The final vector, designated pACYC-CR3022-CR3014, was transfected into E. coli BL21 (DE3). Resultant phage produced in E. coli expressing both the gpD-CR3022 and the gpD-CR3014 represent super-antibodies and are designated ATHR-B4.

Expression and isolation of ATHR-B4, as well as removal of endotoxin was as above described for ATHR-B5 and ATHR-B6.

ATHR-B4 were biochemically characterized. The particle count was established using the NanoSight NS300 Instrument (Malvern Panalytical) with nanoparticle tracking analysis (NTA) software to establish the size and count of bionanoparticles. Dot blots, Western blots and sandwich ELISAs with antibodies against human antibody sequences were used to establish expression of the scFv on the surface of the phage. The functionality of the displayed scFv was demonstrated by its binding to recombinant Spike-RBD protein by ELISA.

### Example 3

In this example, a sequence covering amino acids 19-605 of the natural human ACE2 protein (Accession # BAB40370) were included in a fusion protein of an engineered bacteriophage.

DNA encoding a fusion protein was engineered such that the DNA sequence was attached to the 3′ end of DNA encoding the lambda phage gpD protein with a short piece of DNA in between which codes for the amino acid sequence, GGSGPVGPGGSGAS (SEQ ID NO:2). The engineered protein was expressed in bacteria simultaneously with lambda infection of the same bacterium. These bacteria produced new lambda phage which incorporated the gpD-linker-ACE2 protein together with natural phage gpD. The sequence of the expressed protein is shown below:

| SEQ ID NO: 5 | | |
|---|---|---|
| 1 | MTSKETFTHYQPQGNSDPAHTATAPGGLSAKAPAMTPLMLDTSSRKLVAW | 50 |
| 51 | DGTTDGAAVGILAVAADQTSTTLTFYKSGTFRYEDVLWPEAASDETKKRT | 100 |
| 101 | AFAGTAISIVGGSGPVGPGGSGASSTIEEQAKTFLDKENHEAEDLFYQSS | 150 |
| 151 | LASWNYNTNITEENVQNMNNAGDKWSAFLKEQSTLAQMYPLQEIQNLTVK | 200 |
| 201 | LQLQALQQNGSSVLSEDKSKRLNTILNTMSTIYSTGKVCNPDNPQECLLL | 250 |
| 251 | EPGLNEIMANSLDYNERLWAWESWRSEVGKQLRPLYEEYVVLKNEMARAN | 300 |
| 301 | HYEDYGDYWRGDYEVNGVDGYDYSRGQLIEDVEHTFEEIKPLYEHLHAYV | 350 |
| 351 | RAKLMNAYPSYISPIGCLPAHLLGDMWGREWTNLYSLTVPFGQKPNIDVT | 400 |
| 401 | DAMVDQAWDAQRIFKEAEKFFVSVGLPNMTQGFWENSMLTDPGNVQKAVC | 450 |
| 451 | HPTAWDLGKGDFRILMCTKVTMDDFLTAHHEMGHIQYDMAYAAQPFLLRN | 500 |
| 501 | GANEGFHEAVGEIMSLSAATPKHLKSIGLLSPDFQEDNETEINFLLKQAL | 550 |
| 551 | TIVGTLPFTYMLEKWRWMVFKGEIPKDQWMKKWWEMKREIVGVVEPVPHD | 600 |
| 601 | ETYCDPASLFHVSNDYSFIRYYTRTLYQFQFQEALCQAAKHEGPLHKCDI | 650 |
| 651 | SNSTEAGQKLFNMLRLGKSEPWTLALENVVGAKNMNVRPLLNYFEPLFTW | 700 |
| 701 | LKDQNKNSFVG | 711 |

Where amino acids 1-110 encode for gpD, amino acids 111-124 (italic) encode for a linker (SEQ ID NO: 2), amino acids 125-711 (bold) encode for the ACE2 (19-605).

The nucleic acid sequence for the gpD-ACE2 fusion protein was cloned into the expression vector pACYCDuet-1™ (Novagen) in the first multi-cloning site at the restriction site for NcoI. The resultant plasmid was sequenced across all junctions to ensure proper construction. Expression was under a T7 promoter. The final vector, designated pACYC-ACE2, was transfected into E. coli BL21 (DE3). Resultant phage produced in E. coli expressing the gpD-ACE2 represent super-binders and are designated ATHR-B1. This produced a multivalent receptor mimic with potentially greater affinity/avidity for SARS-CoV-2 over the natural cell surface ACE2 receptor.

ATHR-B1 was expressed, isolated and endotoxin was removed as detailed in example 1.

ATHR-B1 were biochemically characterized. The particle count was established using the NanoSight NS300 Instrument (Malvern Panalytical) with nanoparticle tracking analysis (NTA) software to establish the size and count of bionanoparticles. Dot blots, Western blots and sandwich ELISAs with antibodies against ACE2 were used to establish expression on the surface of the phage. The functionality of the displayed ACE2 was demonstrated by its binding to recombinant Spike-RBD protein by ELISA.

### Example 4

The protein neuropilin 1 (NRP1) was displayed on the surface of an engineered phage λ. NRP1 has been shown to be another possible receptor for the Spike protein of SARS-CoV-2 on the surface of host cells. In this example, we displayed a sequence covering amino acids 273-427 of the natural human NRP1 protein (Accession # NP_001231902).

DNA encoding a fusion protein was engineered such that the DNA sequence was attached to the 3′ end of DNA encoding the lambda phage gpD protein with a short piece of DNA in between which codes for the amino acid sequence, GGSGPVGPGGSGAS (SEQ ID NO: 2). The engineered protein was expressed in bacteria simultaneously with lambda infection of the same bacterium. These bacteria produced new lambda phage which incorporated the gpD-linker-NRP1 protein together with natural phage gpD. The expressed sequence is shown below:

| SEQ ID NO: 6 | | |
|---|---|---|
| 1 | MTSKETFTHYQPQGNSDPAHTATAPGGLSAKAPAMTPLMLDTSSRKLVAW | 50 |
| 51 | DGTTDGAAVGILAVAADQTSTTLTFYKSGTFRYEDVLWPEAASDETKKRT | 100 |
| 101 | AFAGTAISIVGGSGPVGPGGSGASFKCMEALGMESGEIHSDQITASSQYS | 150 |
| 151 | TNWSAERSRLNYPENGWTPGEDSYREWIQVDLGLLRFVTAVGTQGAISKE | 200 |
| 201 | TKKKYYVKTYKIDVSSNGEDWITIKEGNKPVLFQGNTNPTDVVVAVFPKP | 250 |
| 251 | LITRFVRIKPATWETGISMRFEVYGCKIT | 279 |

Where amino acids 1-110 encode for gpD, amino acids 111-124 (italic) encode for a linker (SEQ ID NO: 2), amino acids 125-279 (bold) encode for the NRP1 (273-427). The nucleic acid sequence for the gpD-NRP1 fusion protein was cloned into the expression vector pACYCDuet-1™ (Novagen) in the second multi-cloning site at the restriction site for NdeI. The resultant plasmid was sequenced across all junctions to ensure proper construction. Expression was under a T7 promoter. The final vector, designated pACYC-NRP1 was transfected into E. coli BL21 (DE3). Resultant phage produced in E. coli expressing the gpD-NRP1 represent super-binders and are designated ATHR-B2. This produced a multivalent receptor mimic with potentially greater affinity/avidity for SARS-CoV-2 over the natural cell surface NRP1 receptor.

ATHR-B2 was expressed, isolated and endotoxin was removed as detailed in example 1.

ATHR-B2 were biochemically characterized. The particle count was established using the NanoSight NS300 Instrument (Malvern Panalytical) with nanoparticle tracking analysis (NTA) software to establish the size and count of bionanoparticles. Dot blots, Western blots and sandwich ELISAs with antibodies against NRP1 were used to establish expression on the surface of the phage. The functionality of the displayed NRP1 was demonstrated by its binding to recombinant Spike-RBD protein by ELISA.

This produced a multivalent receptor mimic with potentially greater affinity/avidity for SARS-CoV-2 over the natural cell surface receptor.

### Example 5

In this example, both ACE2 and NRP1 peptides were displayed simultaneously in the same fashion described in examples 4 and 5.

The expression vector was designed as above in example 4 & 5 except that both the gpD-ACE2 and the gpD-NRP1 sequences were placed into the same vector with the gpD-ACE2 cloned into the NcoI site and the gpD-NRP1 cloned into the NdeI site. The final vector, designated pACYC-ACE2-NRP1, was transfected into E. coli BL21 (DE3). Resultant phage produced in E. coli expressing both the gpD-ACE2 and the gpD-NRP1 represent super-binders and are designated ATHR-B3.

Expression and isolation of ATHR-B3, as well as removal of endotoxin was as above described for ATHR-B1 and ATHR-B2.

ATHR-B3 were biochemically characterized. The particle count was established using the NanoSight NS300 Instrument (Malvern Panalytical) with nanoparticle tracking analysis (NTA) software to establish the size and count of bionanoparticles. Dot blots, Western blots and sandwich ELISAs with antibodies against ACE2 and NRP1 were used to establish expression on the surface of the phage. The functionality of the displayed proteins was demonstrated by its binding to recombinant Spike-RBD protein by ELISA.

### Example 6

Following the design of examples 1-5, the two scFv, CR3014 and CR3022, and the two binding proteins, ACE2 and NRP1 were all displayed simultaneously on the surface of a phage λ. In this example a second vector pCOLA was used in place of pACYC for the expression of CR3014 and CR3022. Cloning into pCOLA was the same as in pACYC as these vectors have the same multi-cloning sites and differ primarily only in the drug resistance marker. All other procedures and characterization was the same.

### Example 7

In order to prevent immunogenicity of an engineered bacteriophage as described, human IL-10 was also displayed on the surface of phage A simultaneously with each of the constructs described in examples 1-6.

DNA encoding the human IL-10 sequence (amino acids 19-178, accession # NP_000563) was engineered such that the DNA sequence was attached to the 3′ end of DNA encoding the lambda phage gpD protein with a short piece of DNA in between which codes for the amino acid sequence, GGSGPVGPGGSGAS (SEQ ID NO: 2). The engineered protein was expressed in bacteria with other proteins to be displayed simultaneously with lambda infection of the same bacterium. These bacteria produced new lambda phage which incorporate the gpD-linker-IL-10 protein together with natural phage gpD.

Because IL-10 is known to function as a homodimer which forms an anti-parallel structure, a second construct was made by inserting the IL-10 gene at the 5′ end of the DNA encoding gpD the lambda phage gpD protein. In this case, a methionine residue was included at the N-terminus of the IL-10, followed by the GGSGPVGPGGSGAS (SEQ ID NO: 2) linker which was then followed by the gpD protein.

The two forms of IL-10, gpD-IL-10 and IL-10-gpD were cloned into the pCDFDuet-1™ (Novagen) expression vector at the NcoI and/or NdeI sites respectively. They were expressed separately, as well as together on the surface of phage, as well as in conjunction with the constructs described in examples 1-6.

While certain embodiments of the disclosed subject matter have been described using specific terms, such description is for illustrative purposes only, and it is to be understood that changes and variations may be made without departing from the spirit or scope of the subject matter.

What is claimed is:

1. An engineered bacteriophage comprising:
a first fusion coat protein comprising a first exogenous polypeptide directly or indirectly fused to a first coat protein of the bacteriophage, the first exogenous polypeptide comprising a first specific binding agent for a first proteinaceous target involved in a first disease; and
a second fusion coat protein comprising a second exogenous polypeptide directly or indirectly fused to a second coat protein of the bacteriophage, the second exogenous polypeptide comprising a second specific binding agent for a second proteinaceous target involved in a second disease; wherein
the engineered bacteriophage is free of nucleic acid sequences encoding the first exogenous polypeptide and the second exogenous polypeptide; and wherein the first exogenous polypeptide differs from the second exogenous polypeptide and/or the first coat protein differs from the second coat protein and/or the first disease differs from the second disease.

2. The engineered bacteriophage of claim 1, wherein the first coat protein and the second coat protein are selected from the group consisting of gpD, gpE, gpC, pVIII, pIII, pVI, pVII, pIX, gp23, gp24, gp10A, gp10B, gpF, and gpG coat proteins.

3. The engineered bacteriophage of claim 1, wherein the first binding agent and the second binding agent are the same as one another, or bind different regions of the same protein, or bind different proteins.

4. The engineered bacteriophage of claim 1, wherein the first proteinaceous target and the second proteinaceous target are independently selected from human coronavirus 229E surface glycoprotein or a receptor thereof, human coronavirus NL63 S protein or a receptor thereof, human coronavirus HKU1 spike glycoprotein or a receptor thereof, human coronavirus 0C43 S protein or a receptor thereof, Middle East respiratory syndrome-related coronavirus S protein or a receptor thereof, SARS coronavirus Urbani S protein or a receptor thereof, and severe acute respiratory syndrome coronavirus surface glycoprotein or a receptor thereof.

5. The engineered bacteriophage of claim 1, wherein the first binding agent and/or the second binding agent comprises a neutralizing single-chain antibody, an antibody fragment, a mimic of a cellular receptor, or a nanobody.

6. The engineered bacteriophage of claim 1, wherein the disease is caused by an infectious pathogen selected from a bacterium, a fungus, a parasite, or a virus, or wherein the disease is an auto-immune disease.

7. The engineered bacteriophage of claim 1, wherein the first binding agent binds a protein of a pathogen, or wherein the first binding agent binds a receptor of the protein of the pathogen.

8. The engineered bacteriophage of claim 1, further comprising one or more additional exogenous polypeptides, the one or more additional exogenous polypeptides being directly or indirectly fused to one or more additional coat proteins of the bacteriophage as one or more additional fusion coat proteins and/or the one or more additional exogenous polypeptides being directly or indirectly fused to the first coat protein or the second coat protein.

9. The engineered bacteriophage of claim 1, the first exogenous polypeptide comprising human IL-10 or a fragment thereof.

10. A method for forming an engineered bacteriophage, the method comprising:
transfecting a bacterial cell with one or more expression plasmids, the one or more expression plasmids including a first nucleic acid sequence that encodes a first fusion coat protein and a second nucleic acid sequence that encodes a second fusion coat protein, the first fusion coat protein including a first exogenous polypeptide directly or indirectly fused to a first coat protein and the second fusion coat protein including a second exogenous polypeptide directly or indirectly fused to a second coat protein, the first exogenous polypeptide comprising a first specific binding agent for a first proteinaceous target involved in a disease, and the second exogenous polypeptide comprising a second specific binding agent for a second proteinaceous target involved in a disease, the one or more expression plasmids comprising regulatory sequences such that the first and second fusion coat proteins are transiently expressed by the bacterial cell following the transfection; and
infecting the bacterial cell with a bacteriophage; wherein
the first exogenous polypeptide differs from the second exogenous polypeptide, and/or the first coat protein differs from the second coat protein, and/or the first disease differs from the second disease; and wherein
upon the transfection and the infection, the engineered bacteriophage is produced by the bacterial cell, the engineered bacteriophage including the first fusion coat protein and the second fusion coat protein with the first and second exogenous polypeptides at a surface of the bacteriophage; and wherein
the engineered bacteriophage is free of nucleic acid sequences encoding the first exogenous polypeptide and the second exogenous polypeptide.

11. The method of claim 10, wherein the one or more expression plasmids include a single expression plasmid that includes the first nucleic acid sequence and the second nucleic acid sequence or wherein the one or more expression plasmids includes a first expression plasmid that includes the first nucleic acid sequence and includes a second expression plasmid that includes the second nucleic acid.

12. The method of claim 10, wherein the regulator sequences comprise a first promoter driving expression of the first fusion coat protein and a second promoter driving expression of the second fusion coat protein, and wherein the first promoter and the second promoter are the same promoter or are different promoters.

13. The method of claim 12, wherein the first promoter and the second promoter are independently selected from an inducible promoter and a native phage promoter.

14. The method of claim 12, wherein the first promoter and the second promoter have different strengths from one another.

15. The method of claim 10, wherein the bacteriophage has been modified such that expression of the wild-type first coat protein and/or the wild-type second coat protein has been silenced.

16. A therapeutic composition comprising an engineered bacteriophage, the engineered bacteriophage including a first fusion coat protein, the first fusion coat protein including a first exogenous polypeptide directly or indirectly fused to a first bacteriophage coat protein, the first exogenous polypeptide comprising a first specific binding agent for a first proteinaceous target involved in a first disease, wherein the immunogenic engineered bacteriophage is free of a nucleic acid sequence encoding the first exogenous peptide.

17. The therapeutic composition of claim 16, the engineered bacteriophage comprising a second fusion coat protein, the second fusion coat protein including a second exogenous polypeptide directly or indirectly fused to a second bacteriophage coat protein, the second exogenous polypeptide comprising a second specific binding agent for a second proteinaceous target involved in a second disease, wherein the first exogenous polypeptide differs from the second exogenous polypeptide and/or the first coat protein differs from the second coat protein and/or the first disease differs from the second disease.

18. The therapeutic composition of claim 17, wherein the first binding agent and the second binding agent are the same as one another, or bind different regions of the same protein the same protein, or bind different proteins.

19. The therapeutic composition of claim 16, wherein the first disease and/or the second disease is a coronavirus, influenza, HIV, HCV, HBV, HPV, dengue, Chikungunya, or a West Nile virus.

20. The therapeutic composition of claim 19, wherein the virus is a coronavirus selected from the group consisting of SARS-CoV-1, SARS-Cov-2, MERS, HKU, NL63, HUU1, OC43 and 229E.

21. The therapeutic composition of claim 14, wherein the therapeutic composition is configured for delivery via intramuscular, intravenous, subcutaneous, intradermal, inhalation, absorption, ingestion, or parenteral delivery.


SEQUENCE LISTING (6 sequences)

SEQ ID NO: 1 - protein, 15 aa, Artificial Sequence
  Description of Artificial Sequence Synthetic peptide
GGGGSGGGGSGGGGS

SEQ ID NO: 2 - protein, 14 aa, Artificial Sequence
  Description of Artificial Sequence Synthetic peptide
GGSGPVGPGGSGAS

SEQ ID NO: 3 - protein, 364 aa, Artificial Sequence
  Description of Artificial Sequence Synthetic polypeptide
MTSKETFTHYQPQGNSDPAHTATAPGGLSAKAPAMTPLMLDTSSRKLVAWDGTTDGAAVGILAVAADQTSTTLTFYKSGTFRYEDVLWPEAASDETKKRTAFAGTAISIVGGSGPVGPGGSGASEVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCARGISPFYFDYWGQGTLVTVSSGGGGSGGGGSGGGGSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 4 - protein, 371 aa, Artificial Sequence
  Description of Artificial Sequence Synthetic polypeptide
MTSKETFTHYQPQGNSDPAHTATAPGGLSAKAPAMTPLMLDTSSRKLVAWDGTTDGAAVGILAVAADQTSTTLTFYKSGTFRYEDVLWPEAASDETKKRTAFAGTAISIVGGSGPVGPGGSGASQMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIK

SEQ ID NO: 5 - protein, 711 aa, Artificial Sequence
  Description of Artificial Sequence Synthetic polypeptide
MTSKETFTHYQPQGNSDPAHTATAPGGLSAKAPAMTPLMLDTSSRKLVAWDGTTDGAAVGILAVAADQTSTTLTFYKSGTFRYEDVLWPEAASDETKKRTAFAGTAISIVGGSGPVGPGGSGASSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVG

SEQ ID NO: 6 - protein, 279 aa, Artificial Sequence
  Description of Artificial Sequence Synthetic polypeptide
MTSKETFTHYQPQGNSDPAHTATAPGGLSAKAPAMTPLMLDTSSRKLVAWDGTTDGAAVGILAVAADQTSTTLTFYKSGTFRYEDVLWPEAASDETKKRTAFAGTAISIVGGSGPVGPGGSGASFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKIT